(19) United States
(12) Patent Application Publication
(10) Pub. No.: US 2025/0127636 A1
(43) Pub. Date: Apr. 24, 2025

(54) Classification of Brain Activity Signals

(71) Applicant: Cogitat Ltd., Royston (GB)

(72) Inventors: Barmpas KONSTANTINOS, Royston (GB); Yannis PANAGAKIS, Royston (GB); Dimitrios ADAMOS, Royston (GB); Nikolaos LASKARIS, Royston (GB); Stefanos ZAFEIRIOU, Royston (GB)

(21) Appl. No.: 18/835,771

(22) PCT Filed: Jan. 19, 2023
(86) PCT No.: PCT/GB2023/050092

§ 371 Date: Aug. 5, 2024

(30) Foreign Application Priority Data
Feb. 7, 2022 (GR) 20220100122
Feb. 18, 2022 (GB) 2202239.6

(51) Int. Cl. A61F 2/72; A61B 5/00; A61B 5/245; A61B 5/374; A61F 2/70

(52) U.S. Cl. CPC A61F 2/72; A61B 5/245; A61B 5/374; A61B 5/725; A61B 5/726; A61B 5/7267; A61F 2002/704

(57) ABSTRACT

A computer implemented method of classifying brain activity signals includes: receiving, as input to a neural network, input data comprising a plurality of brain activity signals; applying a first block to the input data to generate a plurality of first order wavelet scalograms, wherein the first convolutional block is configured to apply a plurality of Gabor filters to each of the plurality of brain activity signals, wherein each Gabor filter is associated with a learned bandwidth and learned frequency; applying one or more further blocks to the plurality of first order wavelet scalograms to generate a plurality of feature maps, wherein each further block comprises one or more convolutional layers; and applying a classification block to the plurality of feature maps, wherein the classification block is configured to generate one or more classifications of the plurality of brain activity signals from the plurality of feature maps.

### FIELD

This specification describes systems, apparatus and methods for classifying brain activity signals, such as electroencephalography (EEG) signals, using machine-learning based techniques.

### BACKGROUND

Brain-computer interfaces (BCIs) enable a direct communication of the brain with the external world, using one's neural activity. In recent years, Convolutional Neural Networks (CNNs) have been widely used to perform automatic feature extraction and classification in various electroencephalography (EEG) based tasks. However, their undeniable benefits are counterbalanced by the lack of interpretability properties, the large number of parameters required, as well as the inability to perform sufficiently when only limited amount of data is available.

### SUMMARY

According to a first aspect of this specification, there is described a computer implemented method of classifying brain activity signals. The method comprises: receiving, as input to a neural network, input data comprising a plurality of brain activity signals; applying a first block to the input data to generate a plurality of first order wavelet scalograms, wherein the first convolutional block is configured to apply a plurality of Gabor filters to each of the plurality of brain activity signals, wherein each Gabor filter is associated with a learned bandwidth and learned frequency; applying one or more further blocks to the plurality of first order wavelet scalograms to generate a plurality of feature maps, wherein each further block comprises one or more convolutional layers; and applying a classification block to the plurality of feature maps, wherein the classification block is configured to generate one or more classifications of the plurality of brain activity signals from the plurality of feature maps.

The method may further comprise controlling an apparatus based on the classification of the plurality of brain activity signals. The apparatus may comprise an artificial limb.

According to a further aspect of this specification, there is described a computer implemented method of training a neural network for brain activity signal classification. The method comprises: for each of a plurality of training examples, each comprising a plurality of brain activity signals and one or more ground truth classifications: inputting the plurality of brain activity signals into the neural network; and processing the plurality of brain activity signals through a plurality of blocks of the neural network to generate one or more candidate classifications of the plurality of brain activity signals; updating parameters of the neural network in dependence on a comparison between the candidate classifications and corresponding ground truth classifications, wherein the comparison is performed using a classification objective function. The neural network comprises: a first convolutional block configured to apply a plurality of Gabor filters to each of the plurality of brain activity signals to generate a plurality of first order wavelet scalograms, wherein each Gabor filter is associated with parameters comprising a bandwidth and a frequency; one or more further convolutional blocks configured to generate a plurality of feature maps from the plurality of first order wavelet scalograms, each further convolutional block comprising one or more convolutional layers and associated with a plurality of parameters; a classification block configured to generate one or more classifications of the plurality of brain activity signals from the plurality of feature maps.

The method may further comprise initialising the frequency parameters of the plurality of Gabor filters at different values in a range encompassing an alpha band, a beta band and/or a lower gamma band. The frequency parameters of the plurality of Gabor filters may be initialised at evenly spaced values in the range.

These and other aspects of this specification may further include one or more of the following features, alone or in combination with one or more of the other features.

The first block and/or one or more of the further blocks may be further configured to apply a non-linear function.

The one or more further blocks may comprise a time-frequency convolution block configured to apply a set of temporal convolutional filters in a temporal dimension and a set of frequency convolutional filters in a frequency dimension to each of the first order scalograms to generate a plurality of features for each brain activity signal.

The one or more further blocks may comprise a temporal filtering block configured to apply one or more temporal filters in the temporal dimension to the plurality of feature maps for each brain activity signal.

The one or more further blocks may comprise a spatial filtering block configured to apply one or more spatial convolutions across brain activity signal channels. The spatial filtering block may be configured to output the plurality of feature maps.

One or more of the further convolutional blocks may comprise a pooling layer.

Each Gabor filter, ψ_{λ}, may be of the form:

$${ψ}_{λ} ( t ) = \frac{1}{\sqrt{2 π σ}} {e}^{- {t}^{2} / 2 {σ}^{2}} cos ( 2 π λ t )$$

where t denotes time, 1/σ denotes bandwidth and λ denotes a frequency.

The one or more classifications of the plurality of brain activity signals may comprise: a classification of a resting or active state; a classification of a dynamic state triggered by/underlying the physical or imaginary movement of extremities; a classification of a dynamic state triggered by/underlying a conscious or non-conscious cognitive process related to attention tasks, perception tasks, planning tasks, memory tasks, language tasks, arithmetic tasks, reading tasks, control interface tasks, and specialized tasks like flight or driving, either in a simulator or in a real vehicle action; a classification of an affective state; a classification of an anomaly; a classification of a control intention for an external device; and/or a classification of clinical states.

The brain activity signals may be EEG and/or MEG signals.

According to a further aspect of this specification, there is described a system comprising one or more processors and a memory, the memory storing computer readable instructions that, when executed by the one or more processors, causes the system to perform any one or more of the methods described herein.

The system may further comprise an artificial limb. The system may be configured to control the artificial limb in dependence on the classification of the plurality of brain activity signals.

According to a further aspect of this specification, there is described a computer readable medium storing computer readable instructions that, when executed by a computing system, causes the system to perform any one or more of the methods disclosed herein.

### BRIEF DESCRIPTION OF THE DRAWINGS

FIG. 1 shows a schematic overview of an example method of classifying brain activity signals;

FIG. 2 shows a schematic overview of the operation of an example neural network for classifying brain activity signals;

FIG. 3 shows an example of the operation of a joint temporal-frequency block of a neural network for classifying brain activity signals;

FIG. 4 shows an example structure of a neural network for use in brain signal classification;

FIG. 5 shows a flow diagram of an example method for classifying brain activity signals;

FIG. 6 shows a schematic overview of a method 600 of training a neural network for brain activity signal classification;

FIG. 7 shows a flow diagram of an example method of training a neural network for brain activity signal classification; and

FIG. 8 a schematic example of a computer system/apparatus for performing any of the methods described herein.

### DETAILED DESCRIPTION

Patterns of brain activity are traditionally associated with different brain processes and can be used to differentiate brain states and make behavioural predictions. However, the relevant features are not readily apparent and accessible from brain activity (e.g. EEG) recordings, which may simply record electric potential differences at multiple locations on the skull of a subject.

This specification describes a lightweight, fully-learnable neural network architectures that uses Gabor filters to delocalize signal information into scattering decomposition paths along frequency and slow varying temporal modulations. The network may be used in at least two distinct modelling settings: building either a generic (training across subjects) or a personalized (training within a subject) classifier. Such architectures demonstrate high performance with considerably fewer trainable parameters, as well as shorter training time, when compared to other state-of-the-art deep architectures. Moreover, such network architectures demonstrate enhanced interpretability properties emerging at the level of the temporal filtering operation and enables training of efficient personalized Brain-Computer-Interface (BCI) models with limited amount of training data.

Furthermore, in some embodiments the way in which information from different sensors is combined during its flow through the network can provide a high level of robustness to brain activity sensor malfunctions. Embodiments of the neural networks described herein process each channel of brain activity data separately using depthwise convolutions and capture the spatial filters at the very end of the network, or late in the network. This provides an additional layer of robustness during inference when some of the input brain activity signals are tampered—for example, several EEG sensors of a BCI headset are faulty. The different way in which information from different sensors is combined during its flow through the neural network also allows the use of pre-trained models on dense setups to be adapted quickly and efficiently to lower density sensor arrays while managing to maintain an accuracy close to the original performance.

The lightweight neural network architectures described herein are motivated by the joint time-frequency wavelet scattering transform, with a trainable element introduced that overpasses the fixed wavelets used in the standard wavelet analysis. The joint time-frequency scattering transform and its time-shift invariant properties can capture important underlying characteristics and properties of a brain activity signal. The joint time-frequency wavelet scattering transform, S, consists of a first order time scattering transform on the input signal x(t) using wavelet ψ_{λ}_{₁}(t) followed by a two-dimensional wavelet analysis being carried out independently in the time and frequency domain with two one-dimensional wavelets:

$$Sx = ‖ x ( t ) * {ψ}_{{λ}_{1}} \vert * Ψ ( t , λ ) \vert * ϕ ( t )$$

where Ψ(t, λ)=ψ(t)ψ(λ)^{T} is the product of two one-dimensional wavelets in time and frequency. This equation captures the joint variability of |x(t)*ψ_{λ}_{₁}| in frequency and time, while the modulus and time-averaging operation ϕ(t) ensures time-shift invariance and time-warping stability

Furthermore, some embodiments utilise depthwise convolutions to construct the end-to-end time-frequency scattering transform network efficiently while keeping the number of trainable parameters at a low level. Such neural networks provide enhances interpretability insights into properties of brain signals in the field of motor-imagery compared to “black-box” approaches present in other BCI deep learning networks.

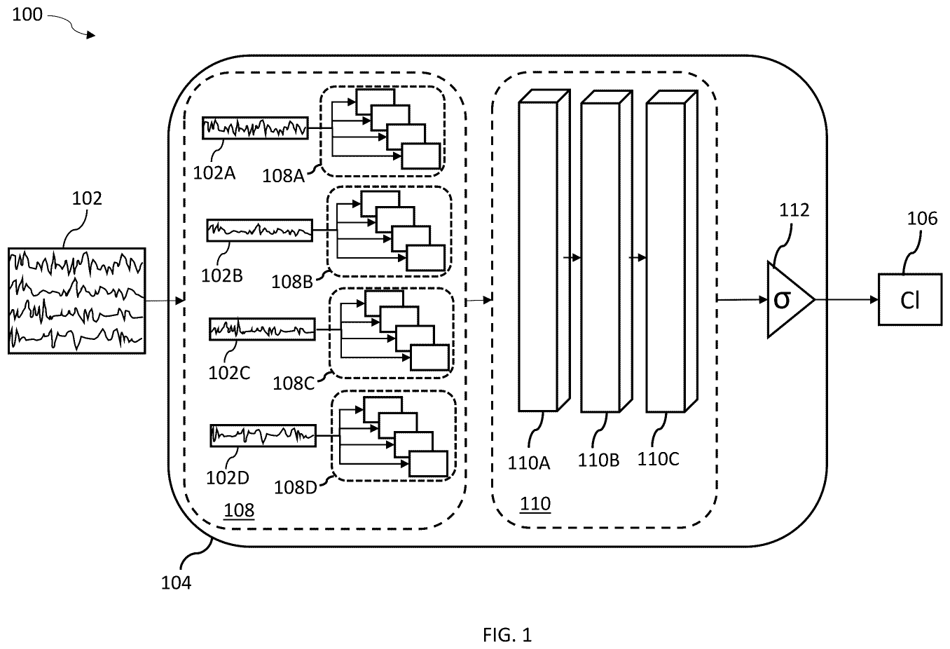

FIG. 1 shows a schematic overview of an example method 100 of classifying brain activity signals 102. A plurality of brain activity signals 102 is input into a neural network 104. The neural network 104 processes the input brain activity signals 102 by implementing a learned joint-scattering transform to generate one or more classifications 106, Cl, of the brain activity signals 102.

The plurality of brain activity signals 102 comprises a plurality of channels of brain activity signals 102A-D. The plurality of brain activity signals is in the time domain. Each channel 102A-D may, for example, correspond to a single electrode/probe of an EEG system and/or magnetometer/probe of an MEG system. Each plurality of brain activity signals 102 input into the neural network 104 may correspond to a fixed-length time window, e.g. 20 seconds of captured EEG data. The brain activity data may be supplied in substantially real time, e.g. streamed from electrodes attached to a subject as the electrodes capture EEG signals.

The neural network 104 comprises a plurality of blocks. A first block 108 is configured to apply a plurality of Gabor filters, {ψ_{λ}(t)}, to each of the channels of brain activity signals 102A-D to generate a plurality of first order wavelet scalograms 108A-D. Each of the Gabor filters is associated with a learned frequency, λ, and bandwidth, 1/σ. A first order wavelet scalogram comprises an amplitude of a corresponding wavelet transform.

The neural network 104 further comprises a one or more further convolutional blocks 110. The one or more further convolutional blocks 110 are configured to generate a plurality of feature maps from the plurality of first order wavelet scalograms 108A-D. The first convolutional block 102A of the one or more further convolutional blocks 110 takes as input the plurality of first order wavelet scalograms 108A-D; subsequent convolutional blocks 102B, 102C take as input the output of a previous convolutional block.

Each further block 110A-C comprises one or more convolutional layers configured to apply a plurality of learned convolutional filters to the input of said convolutional layer. One or more of the convolutional layers may apply convolutional filters in a depthwise manner, i.e. convolutional filters are only applied to a single dimension of the brain activity signals at a time 102, not across multiple dimensions. In some of the convolutional layers, these depthwise convolutions may be applied such that they do not mix brain activity signals between different channels. Consequently, the output of such depthwise convolutions does not mix data from different brain activity signal channels. These convolutional layers may be part of the initial blocks of the further blocks 110A-C.

The neural network 104 further comprises a classification layer 112 (also referred to herein as a “classifier”). The classification layer 112 is configured to determine one or more classifications 106 for the received plurality of brain activity signals 102. The classifier 112 is a parametrised model that provides an output indicative of which class of a plurality of classes the received brain activity signals 102 belong to. For example, the output may be a distribution over a plurality of classes, indicating a probability of the brain activity signals 102 belonging to that class. The parameters of the classifier 112 may also be referred to herein as weights.

In some embodiments, the output classification 106 is an indication of an intended action for an external device, e.g. a classification of a control intention for an external device. This classification 106 may be converted into control signals for controlling the external device to perform the intended action, e.g. control signals for actuators of the device. Examples of such external devices include, but are not limited to, external computing devices, vehicles (either simulated or real) and/or artificial limbs.

In some embodiments, the output classification is a classification of a clinical state and/or a diagnostic classification. Such clinical states/diagnoses may include, for example, Attention deficit hyperactivity disorder, dementia, sleep disorders, Autism Spectrum Disorder or the like. Other examples of potential classifications include, but are not limited to a classification of a resting or active state; a classification of a dynamic state triggered by/underlying the physical or imaginary movement of extremities; a classification of a dynamic state triggered by/underlying a conscious or non-conscious cognitive process related to attention tasks, perception tasks, planning tasks, memory tasks, language tasks, arithmetic tasks, reading tasks, control interface tasks, and specialized tasks like flight or driving, either in a simulator or in a real vehicle action; and/or a classification of an affective state.

The classification layer 112 may comprise a linear layer with an activation function, such as a softmax activation or sigmoid activation. Such a classifier 112 essentially performs logistic regression, and has the advantage that the trained classifier weights can directly be used as importance weights attributed to the features. However, different features can have different distributions, they can be standardized in order to allow for the usage of the regression coefficients directly as an importance measure. The standardization may be performed after the feature module.

It will be appreciated that alternative classifiers 112 may be used, such as one or more neural network layers. In embodiments where the classifier 112 comprise a neural network layer, the neural network layer may be a fully connected neural network layer, a convolutional neural network layer, or the like.

Blocks 108, 110, 112 of the neural network may comprise one or more reshaping layers. Each reshaping is configured to receive a tensor as input and rearrange the components of the input tensor into an output tensor with the same number of components but in a different arrangement/configuration.

Blocks 108, 110, 112 of the neural network may comprise one or more average pooling layers. Each average-pooling layer takes as input a tensor and outputs a smaller tensor comprising averages over a plurality of elements of the input tensor. In effect, an average pooling layer down samples its input by averaging over patches of the input.

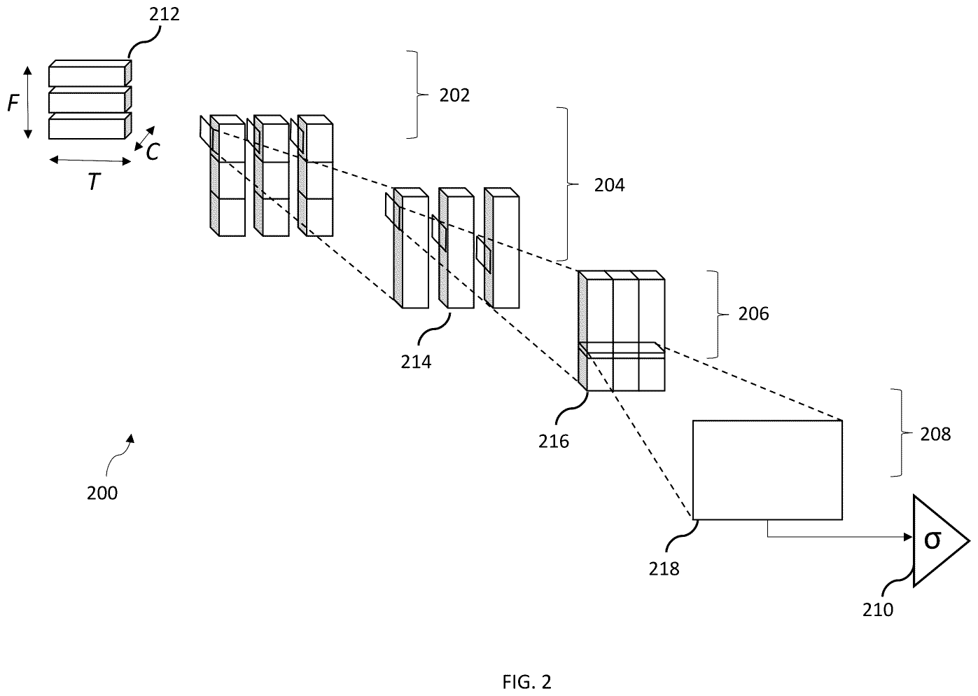

FIG. 2 shows a schematic overview of the operation of an example neural network 200 for classifying brain activity signals. The proposed architecture consists of four main blocks: The first two blocks 202, 204 extract frequency and spatio-temporal information from the brain activity signals through a cascade of learnable wavelet transforms. The third block 206 performs a time-averaging operation to ensure shift invariance while the last block 208 performs the spatial analysis of the signals. Finally a classification block 210 is used to classify the brain activity signals.

The plurality of brain activity signals input into the neural network 200 may be represented as a second order tensor, I, comprising C vectors, x(t)∈ [symbol] ^{T}, representing C channels of brain activity signals (e.g. brain activity signals output by C EEG/MEG probes).

The first block 202 of the neural network computes a 1st order scalogram of the joint time-frequency scattering transform for each channel separately. Symbolically, this may be represented as:

$$X ( λ , t ) = \vert x ( t ) * {ψ}_{λ} ( t ) \vert$$

where x(t)∈ [symbol] ^{T} denotes a one-dimensional input EEG signal, where T is the number of initial EEG time points, and ψ_{λ}(t) denotes a Gabor wavelet/filter. To perform this operation, the raw input signal from each brain activity channel, x(t), may be convolved with a wavelet kernel with size (1, W)=(1, Fₛ/2) where Fₛ is the sampling frequency.

Gabor filters are linear filters comprising a Gaussian kernel function modulated by a sinusoidal plane wave. In some implementations, a Gabor filter may correspond to the function:

$${ψ}_{λ} ( t ) = \frac{1}{\sqrt{2 π σ}} {e}^{- {t}^{2} / ( 2 {σ}^{2} )} cos ( 2 π λ t )$$

where

$$t ∈ [ - \frac{W}{2} , … , \frac{W}{2} ]$$

is time, λ is a (normalised) frequency and 1/σ is a bandwidth. The frequency and bandwidth of each Gabor filter may be learnable parameters of the neural network 104. In some embodiments, λ=Fₙ/Fₛ, where Fₙ denotes the actual frequency and Fₛ is the sampling frequency. In some implementations, the frequency, Fₐ, is restricted to satisfy the Nyquist Theorem 0≤F≤Fₛ/2, which imposes the condition 0≤λ≤½ on the normalised frequency λ.

To implement the first block 202, F such one dimensional Gabor filters are applied to each channel of the brain activity signals. The result of each of these C one-dimensional convolutions is a matrix X(λ, t)∈ [symbol] ^{F×T}. The wavelet filters may be ordered in this matrix based on their normalized frequencies

A non-linear function is applied to the outputs of the Gabor filters. In the example shown, a modulus operation is applied to the elements of this matrix to provide the non-linearity. However, it will be appreciated that other types of non-linearity may alternatively be applied, such as the RELU function, the ELU function or the like. The C matrices are then stacked to produce a 1st order scalogram of all brain activity channels in the form of a three-dimensional Tensor format, X(c, λ, t)∈ [symbol] ^{C×F×T}, 212.

In some embodiments, the first block may comprise an average pooling layer. The average pooling layer may be applied across the temporal dimension to reduce the sampling rate by a predetermined factor, R₁, e.g. by a factor of two or four.

The first block 202 may further comprise one or more reshaping layers, each of which is configured to receive a tensor as input and rearrange the components of the input tensor into an output tensor with the same number of components but in a different arrangement/configuration.

The output of the first block 202 is a three dimensional tensor, X 212, with a first dimension representing the C channels of brain activity signals, a second dimension representing the F Gabor filters, and a third dimension representing time.

The second block 204 of the neural network 200 computes joint spatio-temporal features, F, of the joint scattering transform using convolutional filters. Mathematically, let X(λ, t)∈ [symbol] ^{F×T/4} here be a 1st order scalogram computed in block 1 for one brain activity channel (in this case, after an average pooling operation by a factor of four). The second block 204 computes:

F(λ,t)=|X(λ,t)*Ψ(λ,t)|

where Ψ(λ, t) can be represented as a product of a one-dimensional function of time and a one-dimensional function of frequency, e.g. Ψ(λ, t)=ψ(λ)ψ(t)^{T}.

To perform the convolution operation, depthwise convolutions are utilized to explicitly decouple the relationship within and across the different brain activity channels. Using depthwise operations, first across time and then across frequency (or vice versa), the second block 204 extracts features for each brain activity channel separately, capturing useful spatio-temporal relationships within each channel. The output of the second block is a third order tensor, F, of joint spatio-temporal feature maps 214.

The convolutional filters of the second block 204 are fully trainable. Where average pooling is applied in the first block 202, the convolutional kernels of the second block may have sizes of (1, Fₛ/8) and (Fₛ/8, 1).

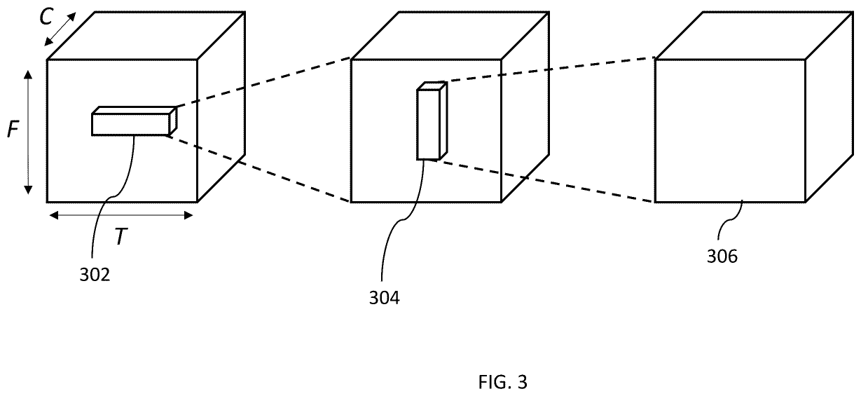

An example of the operation of a second block 204 is shown in further detail in FIG. 3. In the example shown, the convolution across time 302 is applied before the convolution across frequency 304 to generate joint spatio-temporal feature maps 306, though it will be appreciated that these convolutions may alternatively be applied the other way around.

To describe these operations mathematically, the fact that separable convolutions can be obtained by regular convolutions after the application of kernel CP-decomposition is used. Therefore, the three dimensional (3D) tensor X(c, λ, t)∈ [symbol] ^{C×F×T/4} output by the first block can be described as C two-dimensional matrices

$$X ( λ , t ) ∈ {ℝ}^{F \times \frac{T}{R}} .$$

Each kernel of the second block operates into these C matrices X(λ, t) separately to compute the joint spatio-temporal features 306 for the channel:

$$F ( λ , t ) = \vert X ( λ , t ) * Ψ ( λ , t ) \vert = \vert X ( λ , t ) * ψ ( t ) {ψ ( λ )}^{T} \vert$$

The convolution of

$$Ψ ( λ , t ) ∈ {ℝ}^{\frac{{F}_{s}}{8} \times \frac{{F}_{s}}{8}} 1$$

with X(λ, t) may be inserted into the above equation explicitly to give the feature map F(λ, t)∈ [symbol] ^{F×T/R}:

$$F ( λ , t ) = \vert {\sum }_{j = 1}^{{F}_{s} / 8} {\sum }_{i = 1}^{{F}_{s} / 8} Ψ ( j , i ) X ( j + λ , i + t ) \vert$$

A low rank-R CP-decomposition may be applied to the kernel tensor Ψ(λ, t) to rewrite it as:

$$Ψ ( λ , t ) = {u}_{λ} ⊗ {u}_{t} = {u}_{λ} {u}_{t}^{T}$$

By combining the last two equations for rank R=1 (since the operations of block 2 keep the dimensions of the input matrix X intact), the joint spatio-temporal features 306 for the channel can be written as:

$$F ( λ , t ) = \vert {\sum }_{j = 1}^{{F}_{s} / 8} {u}_{λ j} {\sum }_{i = 1}^{{F}_{s} / 8} {u}_{t i}^{T} X ( j + λ , i + t ) \vert$$

This is equivalent to a depthwise convolution across time 302 and a depthwise convolution across frequency 304 (i.e. convolutions across time and frequency that do not mix data across brain activity channels), and consequently the computation of the joint spatio-temporal features 306 can be implemented as convolutional filters, as shown.

In some embodiments, the second block 300 may comprise an average pooling layer (not shown). The average pooling layer may be applied across the temporal dimension to reduce the sampling rate by a predetermined factor, R₂, e.g. by a factor of two or four.

Returning now to FIG. 2, the third block 206 of the neural network 200 performs a temporal filtering/averaging operation of the joint-scattering transform and outputs a temporally filtered features 216, S. The third block 206 applies depthwise convolutions in the temporal dimension, e.g. one-dimensional convolutional filters. The convolutions may, in some embodiments, have a stride greater than one, e.g. a stride of two.

The operation of the third block 206 may be described mathematically for each channel as:

$$\begin{matrix}S ( λ , t ) = F ( λ , t ) * ϕ ( t ) \\ = {\sum }_{{i}^{′} = 1}^{\frac{{F}_{s}}{16}} ϕ ( {i}^{′} ) F ( 1 + λ , {i}^{′} + t ) \\ = {\sum }_{{i}^{′} = 1}^{\frac{{F}_{s}}{16}} ϕ ( {i}^{′} ) \vert {\sum }_{j = 1}^{{F}_{s} / 8} {u}_{λ j} {\sum }_{i = 1}^{{F}_{s} / 8} {u}_{t i}^{T} X ( 1 + j + λ , {i}^{′} + i + t ) \vert\end{matrix}$$

The fourth block 208 of the neural network 200 performs spatial analysis of the signal and generate a set of spatial feature maps 218. Depthwise convolutions are applied in the channel dimension. A depthwise convolution with a kernel of size (C, 1) may be applied to extract the spatial filters of the joint time-frequency scattering transform. Depthwise convolution may be utilized to avoid a mixture of information across different joint time-frequency scattering feature maps.

In some embodiments, each spatial filter may be regularized. For example, each spatial filter may be regularised by using a maximum norm constraint of 1 on its weights, e.g. ∥w∥²<1.

The features 218 output of the fourth block are input into a classifier 210. The classifier 210 processes them to generate data indicative of one or more classifications for the input brain activity signals. The classifier may be a linear classifier. For example, the classifier 210 may comprise a linear layer with sigmoid activation. This classifier essentially performs logistic regression, and has the advantage that the trained classifier weights can directly be used as importance weights attributed to the features.

It will be appreciated that alternative classifiers may be used by the classification module, such as one or more neural networks. In embodiments where the classifier is a neural network, the neural network may be a fully connected neural network, a convolutional neural network, or the like.

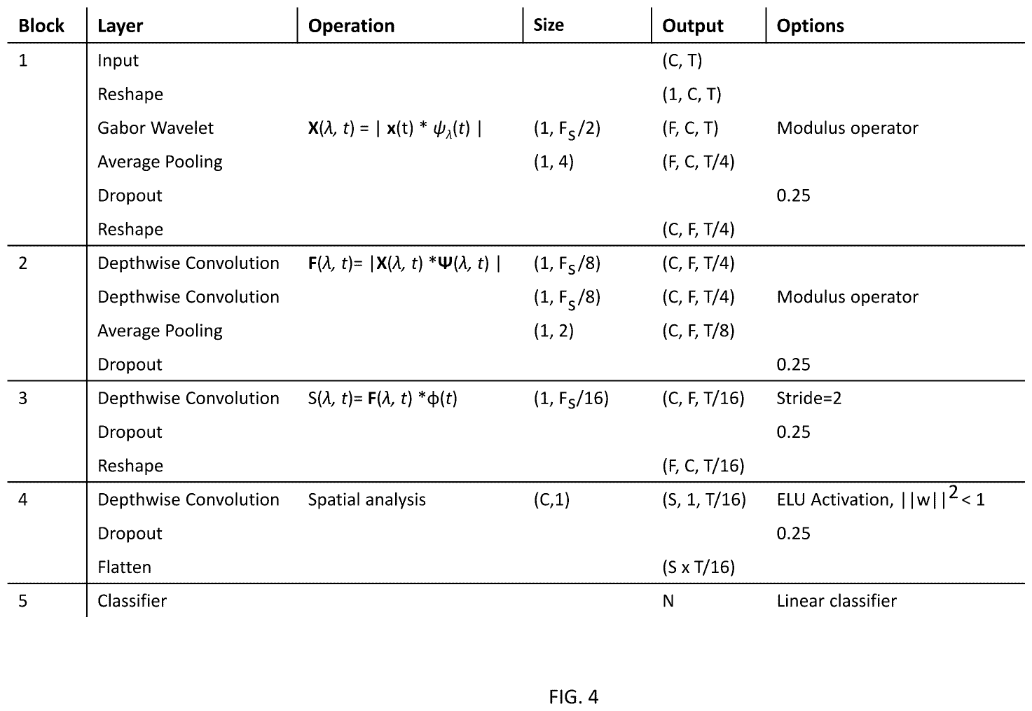

An example structure of a neural network for use in brain signal classification is shown in FIG. 4. The neural network in this example comprises five blocks.

The first block (the “Gabor wavelet block) receives as input brain activity signals in the form of a second order tensor of size (C, T), where C is he number of brain activity signal channels and T is the number of time samples in each channel. A reshape layer is applied to transform the input tensor into a third order tensor of dimension (1, C, T).

A set of learned Gabor filters is then applied to each channel in a Gabor wavelet layer. The set of Gabor filters comprises a set of convolutional filters, each of size (1, Fₛ/2), where Fₛ is the sampling frequency of the brain activity signals. The convolutions are applied in the temporal dimension. Following the convolutions, a non-linear function is applied. In the example shown, the modulus operation is applied. However, it will be appreciated that other types of non-linearity may alternatively be applied, such as the RELU function, the ELU function or the like. The result is a third order tensor of size (F, C, T), where F is the number of Gabor filters applied.

An average pooling layer may then be applied to the output of the Gabor wavelet layer. In the example shown, a filter of size (1, 4) is used to apply the average pooling, which reduces the sampling rate in the temporal dimension by a factor of four. It will be appreciated that other dimensional reduction factors (e.g. 2 or 8) may alternatively be used. The result of this average pooling layer is a third order tensor of dimension (F, C, T/4). The first block ends with a further reshape layer, which swaps the channel and filter dimension of the tensor, resulting in a third order tensor of dimension (C, F, T/4).

During training of the network, a dropout layer may be applied between the average pooling layer and the further reshape layer of the first block. The dropout layer may, for example, have a dropout rate of 0.25. At inference time, this layer may be omitted.

The second block (the “joint time-frequency block”) takes as input the output of the first block (i.e. a tensor of size (C, F, T/4)) and applies a first set of convolutional filters to it, outputting a third order tensor of size (C, F, T/4). The first set of convolutional filters comprises fully learnable convolutional filters. In the example shown, each filter has size (1, Fₛ/8), though it will be appreciated that other sizes may alternatively be used. The first set of convolutional filters is applied in the temporal dimension.

A second set of convolutional filters are applied to the output of the first set of convolutional filters followed by a modulus operation, outputting a third order tensor of size (C, F, T/4). The second set of convolutional filters comprises fully learnable convolutional filters. In the example shown, each filter has size (1, Fₛ/8), though it will be appreciated that other sizes may alternatively be used. The second set of convolutional filters is applied in the frequency dimension (i.e. the filter dimension, F).

An average pooling layer may then be applied. In the example shown, a filter of size (1, 2) is used to apply the average pooling, which reduces the sampling rate in the temporal dimension by a factor of two. It will be appreciated that other dimensional reduction factors (e.g. 4 or 8) may alternatively be used. The result of this average pooling layer is a third order tensor of dimension (C, F, T/8). This is the output of the third bock.

During training of the network, a dropout layer may be applied after the average pooling layer. The dropout layer may, for example, have a dropout rate of 0.25. At inference time, this layer may be omitted.

The third block (the “temporal averaging block”) takes as input the output of the second block (i.e. a tensor of size (C, F, T/8)) and applies a third set of convolutional filters to it, outputting a third order tensor of size (C, F, T/16). The third set of convolutional filters comprises fully learnable convolutional filters. In the example shown, each filter has size (1, Fₛ/16), though it will be appreciated that other sizes may alternatively be used. The third set of convolutional filters is applied in the temporal dimension. It may be applied with a predefined stride, e.g. a stride of two, as shown in the example of FIG. 4.

A reshape layer may then be applied to reshape the output tensor by swapping the frequency and channel dimensions, outputting a tensor of size (F, C, T/16).

During training of the network, a dropout layer may be applied before the reshape layer. The dropout layer may, for example, have a dropout rate of 0.25. At inference time, this layer may be omitted.

The fourth block (the “spatial analysis block”) takes as input the output of the third block (i.e. a tensor of size (F, C, T/16)) and applies a fourth set of convolutional filters to it, outputting a third order tensor of size (S, 1, T/16), where S is the number of filters in the fourth set of convolutional filters. The fourth set of convolutional filters comprises a fully learnable convolutional filters. In the example shown, each filter has size (C, 1), though it will be appreciated that other sizes may alternatively be used. The third set of convolutional filters is applied in the spatial dimension (i.e. along the channel dimension, C).

The convolutional filters are followed by a activation function. In this example, the ELU activation function is used, though it will be appreciated that other activation functions may alternatively be used.

The fourth convolutional layer may be followed by a flattening layer, which converts the (S, 1, T/16) tensor output by the fourth convolutional layer into a vector of dimension (S×T/16).

During training, the magnitude of the weights in this layer may be restricted to have an absolute value of less than one, i.e. ∥w∥²<1 for weights w. The fourth convolutional layer may be followed by a dropout layer. The dropout layer may, for example, have a dropout rate of 0.25. At inference time, this layer may be omitted.

The output of the fourth block is input into a fifth block (the “classification block”), which processes it using a classifier to generate one or more classifications of the input brain activity signals. The output of the classifier may be an N dimensional vector, each component of which provides a score indicative of the brain activity signals belonging to one of N classifications. For example, the output is a distribution over N potential classifications. In the example shown, the classifier is a linear classifier, though it will be appreciated that other types of classifier may alternatively be used (e.g. a fully connected neural network layer).

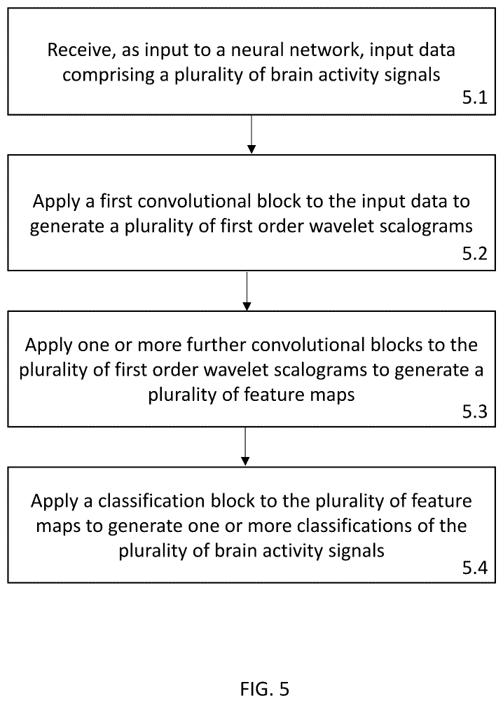

FIG. 5 shows a flow diagram of an example method for classifying brain activity signals. The method may be performed by one or more computers operating in one or more locations.

At operation 5.1, input data comprising a plurality of brain activity signals is received as input to a neural network. The brain activity signals may comprise a plurality of channels, C, of brain activity data in the time domain. Each channel may correspond to the output of one or more EEG and/or MEG probes. Each channel may be in the form of a time series of brain activity signal data comprising T samples. The samples may be taken at a sampling frequency Fₛ.

At operation 5.2, a first convolutional block is applied to the input data to generate a plurality of first order wavelet scalograms. The first convolutional block is configured to apply a plurality of Gabor filters to each of the plurality of brain activity signals (e.g. in the temporal dimension within each channel). Each Gabor filter is associated with parameters comprising a learned bandwidth and a frequency. These parameters are the learnable parameters of the network during training.

At operation 5.3, one or more further convolutional blocks is applied to the plurality of first order wavelet scalograms to generate a plurality of feature maps.

The one or more further convolutional blocks may comprise a time-frequency convolution block. The time-frequency convolution block comprises a first set of convolutional filters that are applied in the temporal dimension. The first set of convolutional filters may be 1D convolutional filters. The time-frequency convolution block further comprises a second set of convolutional filters that are applied in the frequency dimension. The second set of convolutional filters may be 1D convolutional filters.

The one or more further convolutional blocks may further comprise a temporal filtering block configured to a set of temporal filters to the plurality of feature maps for each brain activity signal. The set of temporal filters comprises a set of convolutional filters that are applied in the temporal dimension. The set of temporal filters may be 1D convolutional filters.

The one or more further convolutional blocks may alternatively or additionally comprise a spatial filtering block configured to apply a set of spatial filters across brain activity signals. The set of spatial filters comprises a set of convolutional filters that are applied in the spatial dimension (i.e. across channels of brain activity signals). The output of the spatial filtering block may be the set of feature maps output by the one or more further convolutional blocks.

One or more of the further convolutional blocks may comprise an average pooling layer and/or a reshaping layer.

At operation 5.4, a classification block is applied to the plurality of feature maps to generate one or more classifications of the plurality of brain activity signals. The classification bock may output a score for each of a plurality of potential classifications for the brain activity signals, with the one or more classifications selected based on these scores.

Based on the one or more classifications of the plurality of brain activity signals, an apparatus may be controlled. An artificial limb may be controlled based on the one or more classifications. For example, the one or more classifications may be converted into control signals for use in controlling actuators of the artificial limb.

The one or more classifications of the plurality of brain activity signals comprises: a classification of a resting or active state; a classification of a dynamic state triggered by/underlying the physical or imaginary movement of extremities; a classification of a dynamic state triggered by/underlying a conscious or non-conscious cognitive process related to attention tasks, perception tasks, planning tasks, memory tasks, language tasks, arithmetic tasks, reading tasks, control interface tasks, and specialized tasks like flight or driving, either in a simulator or in a real vehicle action; a classification of an affective state; a classification of an anomaly; a classification of a control intention for an external device; and/or a classification of clinical states.

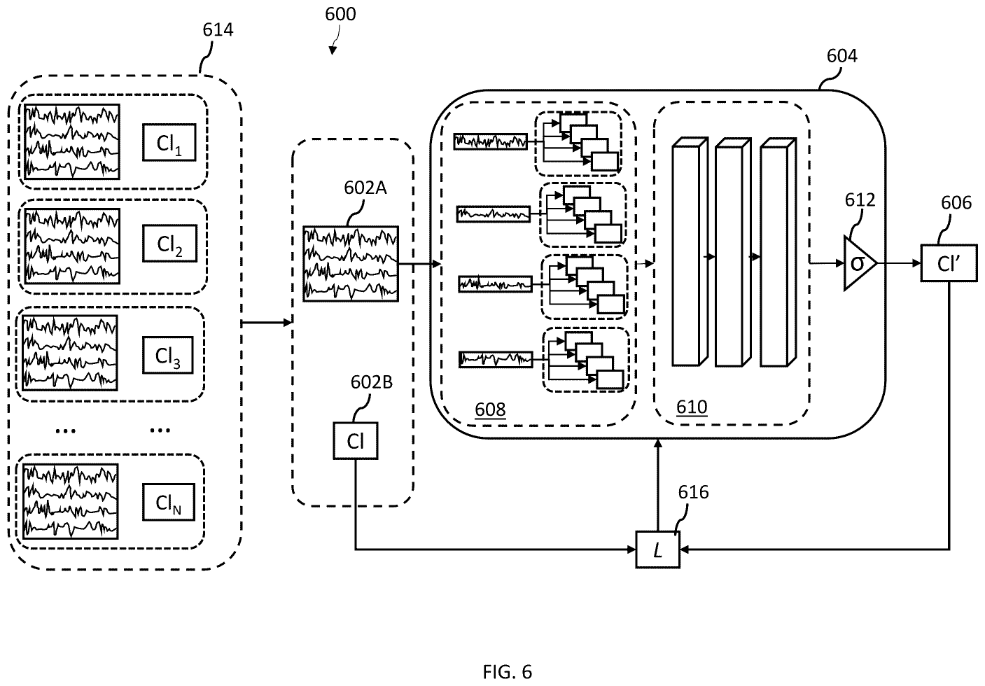

FIG. 6 shows a schematic overview of a method 600 of training a neural network for brain activity signal classification. A training sample 602 comprising a plurality of brain activity signals 602A and a ground truth classification 602B, Cl, is obtained from a training dataset 614 comprising a plurality of training samples 114A-D. The training sample 602 may be taken from a batch/mini-batch of the training dataset 614 each comprising a plurality of training samples forming a proper subset of the training dataset 614. The batch size may, for example, lie in the range [32, 256], for example 64.

The plurality of brain activity signals 602A from a training sample 602 are input into a neural network 604, which processes them based on parameters of the neural network 604 to generate a candidate classification 606, Cl′. The candidate classification 606 is compared to the corresponding ground truth classification 602B using a loss/objective function 616. Updates to parameters of the neural network 604 are determined based on the comparison.

Examples of training datasets 614 include, but are not limited to, the BCI IVa dataset, which comprises brain recordings from five healthy subjects, registered via 118 EEG sensors, while performing a series of randomized cue-triggered motor-imagery tasks. As a further, non-limiting example, the training dataset 614 may be the PhysioNet dataset, which comprises brain recordings from 109 healthy participants, registered via 64 EEG sensors with a sampling frequency of 160 Hz, while performing a series of pseudo-randomized cue-triggered MI tasks. In general, any dataset comprising brain activity signals with known classifications may be used.

The neural network 604 comprises a plurality of blocks. A first block 608 is configured to apply a plurality of Gabor filters to each of the channels of brain activity signals to generate a plurality of first order wavelet scalograms. A further one or more blocks 610 are configured to generate a set of feature maps from the plurality of first order wavelet scalograms. A classification block 612 is configured to apply a classifier to the set of feature maps. The structure and function of blocks are described in more detail above with reference to FIGS. 1-5.

During training, the parameters of the first block (e.g. the bandwidth and frequency of the Gabor filters) may be restricted to satisfy the Nyquist Theorem, e.g.:

$$0 \leq F \leq \frac{{F}_{s}}{2} \to 0 \leq λ \leq \frac{1}{2}$$

Furthermore, in some embodiments the frequency parameters of the first block may be initialised at frequencies corresponding to frequencies in the alpha band [8, 13 Hz], the beta band [13, 40 Hz], and/or the lower gamma band [30, 40 Hz]. For example, during initialization the values for frequency values λ may be evenly spaced in the following range:

$$\frac{8}{{F}_{s}} \leq λ \leq \frac{40}{{F}_{s}}$$

The bandwidth parameters may, in some embodiments, be initialised with the same value for all wavelet filters. The “right” choice of its initial value depends on different datasets and should be treated during the hyperparameter tuning phase of the network.

A reasonable range for the bandwidth σ values may be between [4\sqrt{2 log 2}, 2 W \sqrt{2 log 2}] such that the full-width at half-maximum of the frequency response is within 1/W and ½.

The loss/objective function 616 compares the candidate classification(s) 606 to corresponding ground-truth classifications 602B. Examples of such a loss/objective function 616 include classification losses, such as a cross entropy loss. Other classification losses may alternatively be used, such as an L2 loss (i.e. a mean squared error) between the candidate classification 606 and the ground truth classification 602B.

To determine the parameter updates, an optimisation routine may be applied to the loss/objective function 616. The goal of the optimisation routine may be to minimise or maximise the loss/objective function 616. Examples of such an optimisation routine include (mini-batch) stochastic gradient descent.

In some implementations the Adam optimizer may be used with a batch size of sixty-four, with the goal of minimizing the cross-entropy loss function. The training may be iterated for 150 training iterations.

For example, the Adam optimizer may be used with a learning rate of 0.01 for the first 30 epochs and 0.0001 for the remainder 20 epochs. Batch normalization layers may be introduced between blocks to stabilize training and improve performance.

Alternatively, the Adam optimizer may be used with a learning rate of 0.01 for the first 50 epochs, 0.001 for epochs between 50-80 and 0.0001 for the remainder 20 epochs. A batch normalization layer may be introduced only after the spatial convolutional layer to stabilize training and improve performance.

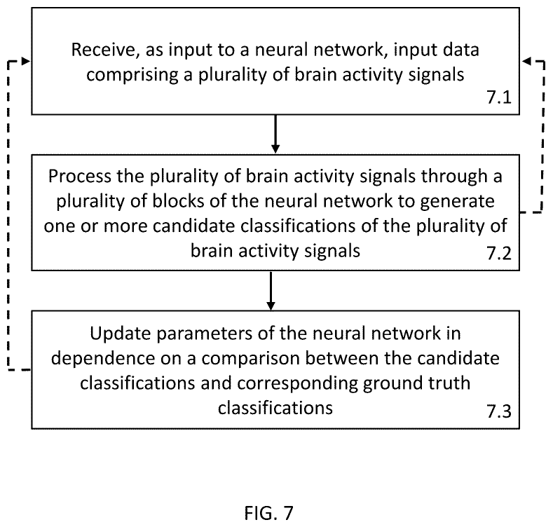

FIG. 7 shows a flow diagram of an example method of training a neural network for brain activity signal classification.

Frequency parameters of a plurality of Gabor filters of the neural network may be initialised at different values in a range encompassing an alpha band and a beta band. The frequency parameters of the plurality of Gabor filters may be initialised at evenly spaced values in the range.

At operation 7.1, input data from a training sample comprising a plurality of brain activity signals is received as input to a neural network. The training sample is obtained from a training dataset comprising a plurality of training samples, each sample comprising a respective plurality of brain activity signals and a corresponding ground-truth classification of the respective plurality of brain activity signals.

At operation 7.2, the plurality of brain activity signals are processed through a plurality of blocks of the neural network to generate one or more candidate classifications of the plurality of brain activity signals.

The plurality of blocks of the neural network comprise a first convolutional block configured to apply a plurality of Gabor filters to each of the plurality of brain activity signals to generate a plurality of first order wavelet scalograms. The plurality of block of the neural network further comprise one or more further convolutional blocks configured to generate a plurality of feature maps from the plurality of first order wavelet scalograms. The plurality of block of the neural network further comprise a classification block configured to generate one or more candidate classifications of the plurality of brain activity signals from the plurality of feature maps. Examples of these blocks are described throughout this specification.

In some embodiments, operations 7.1 and 7.2 may be iterated over a batch/mini-batch of training samples before proceeding to operation 7.3.

At operation 7.3, parameters of the neural network are updated in dependence on a comparison between the candidate classifications and corresponding ground truth classifications. The comparison is performed using an objective function. The objective function may take into account the comparison of candidate and ground truth classifications for a plurality of training samples (e.g. a training batch) when determining each set of updates.

The loss function may be any classification loss function. For example, a cross entropy loss function may be used.

The parameter updates may be determined by applying an optimisation routine to the loss/objective function. For example, stochastic gradient descent may be applied to the loss function to determine the parameter updates. In some embodiments, the Adam optimiser may be used.

Operations 7.1 to 7.3 may be iterated until a threshold condition is satisfied. The threshold condition may be a threshold number of training epochs and/or a threshold performance being reached on a test dataset.

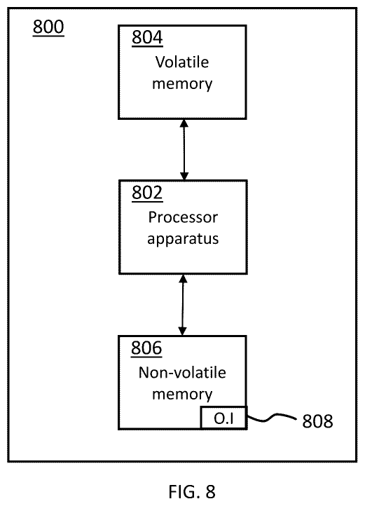

FIG. 8 shows a schematic example of a system/apparatus 800 for performing any of the methods described herein. The system/apparatus shown is an example of a computing device. It will be appreciated by the skilled person that other types of computing devices/systems may alternatively be used to implement the methods described herein, such as a distributed computing system. The system/apparatus 800 may be a distributed system. The system/apparatus may form a part of a brain-computer interface system comprising one or more brain activity probes (e.g. EEG and/or MEG sensors) for sensing brain activity signals and an apparatus controllable based on classification of the sensed brain activity signals.

The apparatus (or system) 6800 comprises one or more processors 802. The one or more processors control operation of other components of the system/apparatus 800. The one or more processors 802 may, for example, comprise a general-purpose processor. The one or more processors 802 may be a single core device or a multiple core device. The one or more processors 802 may comprise a Central Processing Unit (CPU) or a graphical processing unit (GPU). Alternatively, the one or more processors 802 may comprise specialised processing hardware, for instance a RISC processor or programmable hardware with embedded firmware. Multiple processors may be included.

The system/apparatus comprises a working or volatile memory 804. The one or more processors may access the volatile memory 804 in order to process data and may control the storage of data in memory. The volatile memory 804 may comprise RAM of any type, for example Static RAM (SRAM), Dynamic RAM (DRAM), or it may comprise Flash memory, such as an SD-Card.

The system/apparatus comprises a non-volatile memory 806. The non-volatile memory 806 stores a set of operation instructions 808 for controlling the operation of the processors 802 in the form of computer readable instructions. The non-volatile memory 806 may be a memory of any kind such as a Read Only Memory (ROM), a Flash memory or a magnetic drive memory.

The one or more processors 802 are configured to execute operating instructions 808 to cause the system/apparatus to perform any of the methods described herein. The operating instructions 808 may comprise code (i.e. drivers) relating to the hardware components of the system/apparatus 800, as well as code relating to the basic operation of the system/apparatus 800. Generally speaking, the one or more processors 802 execute one or more instructions of the operating instructions 808, which are stored permanently or semi-permanently in the non-volatile memory 806, using the volatile memory 804 to store temporarily data generated during execution of said operating instructions 808.

Implementations of the methods described herein may be realised as in digital electronic circuitry, integrated circuitry, specially designed ASICs (application specific integrated circuits), computer hardware, firmware, software, and/or combinations thereof. These may include computer program products (such as software stored on e.g. magnetic discs, optical disks, memory, Programmable Logic Devices) comprising computer readable instructions that, when executed by a computer, such as that described in relation to FIG. 8, cause the computer to perform one or more of the methods described herein.

Any system feature as described herein may also be provided as a method feature, and vice versa. As used herein, means plus function features may be expressed alternatively in terms of their corresponding structure. In particular, method aspects may be applied to system aspects, and vice versa.

Furthermore, any, some and/or all features in one aspect can be applied to any, some and/or all features in any other aspect, in any appropriate combination. It should also be appreciated that particular combinations of the various features described and defined in any aspects of the invention can be implemented and/or supplied and/or used independently.

Although several embodiments have been shown and described, it would be appreciated by those skilled in the art that changes may be made in these embodiments without departing from the principles of this disclosure, the scope of which is defined in the claims.

1. A computer implemented method of classifying brain activity signals, the method comprising:
receiving, as input to a neural network, input data comprising a plurality of brain activity signals;
applying a first block to the input data to generate a plurality of first order wavelet scalograms, wherein the first convolutional block is configured to apply a plurality of Gabor filters to each of the plurality of brain activity signals, wherein each Gabor filter is associated with a learned bandwidth and learned frequency;
applying one or more further blocks to the plurality of first order wavelet scalograms to generate a plurality of feature maps, wherein each further block comprises one or more convolutional layers; and
applying a classification block to the plurality of feature maps, wherein the classification block is configured to generate one or more classifications of the plurality of brain activity signals from the plurality of feature maps.

2. The method of claim 1, further comprising controlling an apparatus based on the classification of the plurality of brain activity signals.

3. The method of claim 2, wherein the apparatus comprises an artificial limb.

4. A computer implemented method of training a neural network for brain activity signal classification, the method comprising:
for each of a plurality of training examples, each comprising a plurality of brain activity signals and one or more ground truth classifications:
inputting the plurality of brain activity signals into the neural network; and
processing the plurality of brain activity signals through a plurality of blocks of the neural network to generate one or more candidate classifications of the plurality of brain activity signals;
updating parameters of the neural network in dependence on a comparison between the candidate classifications and corresponding ground truth classifications, wherein the comparison is performed using a classification objective function,
wherein the neural network comprises:
a first convolutional block configured to apply a plurality of Gabor filters to each of the plurality of brain activity signals to generate a plurality of first order wavelet scalograms, wherein each Gabor filter is associated with parameters comprising a bandwidth and a frequency;
one or more further convolutional blocks configured to generate a plurality of feature maps from the plurality of first order wavelet scalograms, each further convolutional block comprising one or more convolutional layers and associated with a plurality of parameters;
a classification block configured to generate one or more classifications of the plurality of brain activity signals from the plurality of feature maps.

5. The method of claim 4, further comprising initialising the frequency parameters of the plurality of Gabor filters at different values in a range encompassing an alpha band, a beta band and/or a lower gamma band.

6. The method of claim 5, wherein the frequency parameters of the plurality of Gabor filters are initialised at evenly spaced values in the range.

7. The method of claim 1, wherein the first block and/or one or more of the further blocks is further configured to apply a non-linear function.

8. The method of claim 1, wherein the one or more further blocks comprises a time-frequency convolution block configured to apply a set of temporal convolutional filters in a temporal dimension and a set of frequency convolutional filters in a frequency dimension to each of the first order scalograms to generate a plurality of features for each brain activity signal.

9. The method of claim 8, wherein the one or more further blocks comprises a temporal filtering block configured to apply one or more temporal filters in the temporal dimension to the plurality of feature maps for each brain activity signal.

10. The method of claim 1, wherein the one or more further blocks comprises a spatial filtering block configured to apply one or more spatial convolutions across brain activity signal channels.

11. The method of claim 10, wherein the spatial filtering block is configured to output the plurality of feature maps.

12. The method of claim 1, wherein one or more of the further convolutional blocks comprises a pooling layer.

13. The method of claim 1, wherein each Gabor filter, ψλ, is of the form:
$$ψ\_λ ( t ) = 1 / √ 2 πσ e \hat{} ( - t \hat{} 2 / ( 2 σ \hat{} 2 ) ) cos ( 2 πλ t )$$
where t denotes time, 1/σ denotes bandwidth and λ denotes a frequency.

14. The method of claim 1, wherein the one or more classifications of the plurality of brain activity signals comprises: a classification of a resting
or active state; a classification of a dynamic state triggered by/underlying the physical
or imaginary movement of extremities; a classification of a dynamic state triggered
by/underlying a conscious or non-conscious cognitive process related to attention tasks, perception tasks, planning tasks, memory tasks, language tasks, arithmetic tasks, reading tasks, control interface tasks, and specialized tasks like flight or driving, either in a simulator or in a real vehicle action; a classification of an affective state; a
classification of an anomaly; a classification of a control intention for an external device; and/or a classification of clinical states.

15. The method of claim 1, wherein the brain activity signals are EEG and/or MEG signals.

16. A system comprising one or more processors and a memory, the memory storing computer readable instructions that, when executed by the one or more processors, causes the system to perform the method of claim 1.

17. The system of claim 16, further comprising an artificial limb, wherein the system is configured to control the artificial limb in dependence on the classification of the plurality of brain activity signals.

18. A computer readable medium storing computer readable instructions that, when executed by a computing system, causes the system to perform the method of claim 1.
